(19) United States
(12) Patent Application Publication
(10) Pub. No.: US 2021/0369772 A1
(43) Pub. Date: Dec. 2, 2021

(54) NUTRITION-BASED SUPPORT FOR THE BODY'S OWN WOUND HEALING PROCESSES

(71) Applicant: SANAFACTUR GMBH, Enger (DE)

(72) Inventors: Cathrin LINDENSCHMIDT, Ibbenbueren (DE); Alexander MAASSEN, Muenster (DE)

(73) Assignee: SANAFACTUR GMBH, Enger (DE)

(21) Appl. No.: 17/282,087

(22) PCT Filed: Sep. 26, 2019
(86) PCT No.: PCT/DE2019/100850

§ 371 Date: Apr. 1, 2021

(30) Foreign Application Priority Data
Oct. 1, 2018 (DE) 10 2018 124 227.1

(51) Int. Cl. A61K 33/30; A61K 31/51; A61K 31/525; A61K 31/455; A61K 31/197; A61K 31/4415; A61K 31/519; A61K 31/714; A61K 31/375; A61K 31/405; A61K 33/06; A61K 31/4188; A61K 31/198; A61K 47/42; A61K 47/12; A23L 33/16; A23L 33/175; A23L 33/12; A23L 33/125; A23L 33/15; A23L 33/185; A23L 33/19; A23L 33/18

(52) U.S. Cl. CPC A61K 33/30; A23V 2002/00; A61K 31/525; A61K 31/455; A61K 31/197; A61K 31/4415; A61K 31/519; A61K 31/714; A61K 31/375; A61K 31/405; A61K 33/06; A61K 31/4188; A61K 31/198; A61K 47/42; A61K 47/12; A23L 33/16; A23L 33/175; A23L 33/12; A23L 33/125; A23L 33/15; A23L 33/185; A23L 33/19; A23L 33/18; A61K 31/51

(57) ABSTRACT

The use of a preparation for nutrition-based support of the body's own wound healing processes at the local and systemic level by oral intake of the preparation, which contains per gram as a basic preparation the following mixture of vitamins and nutrients:
a. 330-385 μg of vitamin B1,
b. 420-490 μg of vitamin B2,
c. 4.8-5.6 mg of vitamin B3,
d. 1.8-2.1 mg of vitamin B5,
e. 420-490 μg of vitamin B6,
f. 15-18 μg of vitamin H,
g. 80-390 μg of vitamin B9,
h. 1.0-4.9 μg of vitamin B12,
i. 32-156 mg of vitamin C,
j. 150-731 mg of magnesium,
k. 4-20 mg of zinc and
l. 3-200 mg of tryptophan.

### TECHNICAL FIELD

The invention describes a preparation for oral ingestion that has been tailored to the special requirements of patients with wounds and supports the body's own wound healing processes both locally and systemically.

### BACKGROUND OF THE INVENTION

The human body as a whole and also the organs and structures inside it can only function if they are clearly delimited from each other and from their environment. Wounds represent a failure or loss of this delimitation. The body has therefore developed a complex, multifactorial system of processes for wound healing in order to restore the necessary delimitation between inside and outside and between different body regions.

There are currently numerous products (“wound dressings”) which are intended to promote these healing processes, especially in the case of wounds located on the body surface, by creating an environment conducive to wound healing or are intended to reduce wound concomitants such as, for example, unpleasant odors and to thus improve the patients' quality of life. An extensive range of different wound dressings is available today to the qualified personnel working in wound care, in order to be able to provide good care for the patients according to the particularities of the particular wound.

However, wound healing is not a purely local process at the site of the wound. Successful wound healing requires suitable boundary conditions which have an effect on the entire body. Especially in the case of so-called “secondary” wound healing, this includes the provision of the nutrients required for a more or less extensive tissue regeneration. Patients with relatively large wounds therefore have distinctly increased nutrient requirements compared to comparable healthy patients. Any deficiency in a micronutrient or macronutrient becomes the limiting factor for tissue regeneration and for the wound closure that is striven for.

Accordingly, there are already preparations which have been tailored to the nutrient requirements of general diseases or of wound healing processes by improving the general nutritional situation. Preparations according to U.S. Pat. No. 5,714,472 are aimed at the patient's general nutritional situation. Analogously, EP 2 503 906 B1 describes a protein-rich preparation which is enriched with exogenous nucleotides. WO 2018/091566 A1 describes a preparation which has been tailored to the patients' general nutritional situation and contains polyunsaturated fatty acids, vitamin E, vitamin D and the amino acids glycine, arginine and tryptophan contained in the proteins of the preparation. Said preparation is aimed at various clinical pictures such as COPD and neurological diseases and disorders, the common denominators of which are a reduced food intake due to illness and a generally increased metabolic situation. Accordingly, the goal of the aforementioned preparations with their ingredients is a rather unspecific improvement of the general condition and not the pathophysiological peculiarities of the wound healing processes.

WO 2014/022886 A1 describes a preparation which exhibits particularly positive effects in the case of patients with atopic and nonatopic eczemas. These diseases involve genetic and immunological causes and result in inflammatory reactions which cause malnutrition and which the preparation tries to act on with its ingredients such as, inter alia, glycine, vitamin B6, taurine or magnesium. Even though inflammatory reactions in disorders can play a role in certain phases of wound healing, they are only a minor part of the metabolic pathways involved in wound healing. The fact that zinc addition, mentioned incidentally in this document, has a positive effect on wound healing is well known and described in this generality.

EP 2 196 099 A1 describes a preparation which is primarily aimed at optimizing the patients' immune processes. A mixture of maltodextrin, arginine, omega-3 fatty acids, collagen, vitamins and zinc is considered beneficial for this purpose

EP 0 960 572 A1 describes a preparation which contains arginine, vitamin C, vitamin E, flavonoids and other macro- and micronutrients and is intended to have improved effects on blood circulation and on control of inflammations in addition to improving wound healing.

Still other preparations according to EP 1 543 735 A1 and U.S. Pat. No. 5,733,884 serve to optimize local biochemical processes of wound healing in a specific manner. Said preparations have been attuned to the importance of the amino acids arginine and proline, but additionally also contain macronutrients in the form of proteins, fats and carbohydrates. Both of the aforementioned amino acids act directly on local wound healing processes.

EP 1 633 377 B1 describes a preparation including macronutrients with an increased content of the amino acids leucine and/or glycine. Said preparation aims at stimulating or compensating for inadequate prolidase activity, which plays a role in the synthesis of relevant molecules as building blocks for local tissue regeneration.

The preparation according to WO 2013/106570 A1 is a combination of beta-hydroxy-beta-methylglutamine (HMB), arginine and glutamine and optionally other macronutrients and has been specifically tailored to supporting wound healing for wounds in patients with diabetic ulcers who have a decreased serum albumin level and a reduced circulation of the extremities. However, this only applies to some of the patients; the preparation thus acts in a very specific subgroup of patients with wounds and specifically on certain metabolic pathways in said patients.

The preparation according to WO 97/16079 A1 is aimed at the nutritional requirements of children up to 10 years of age, which—as stated in the document—differ greatly from the requirements of adult patients. The majority of patients with wounds, especially chronic wounds, are, however, of an advanced age. The preparation corresponds to a complete food tailored to children, and a positive effect on wound healing due to addition of certain fatty acids and cysteine-rich protein sources is additionally described in a dependent claim.

Besides the basic supply of macronutrients and/or micronutrients, the aforementioned preparations as prior art thus primarily act either systemically, for example on the immune system, or are intended to support certain aspects of micronutrient supply or of local biochemical wound healing processes. However, wound healing requires body-wide synchronization of both local biochemical synthesis processes for tissue regeneration and systemic processes beyond nutrient provision. The substances contained in the aforementioned preparations therefor are all constituents of common nutritional recommendations and their fundamentally positive effect on wound healing at one level or another has been sufficiently described.

Proceeding from this insight, it has surprisingly been found that, building on a basic supply of the customary micronutrients such as vitamins and minerals to the body, a specific enrichment of the preparation with folate, magnesium, zinc, vitamin B12 and vitamin C promotes, in a particular way and in a coordinated manner, both local and systemic processes beneficial for wound healing and thus supports the process of wound healing. The extent of this positive effect goes far beyond what is normally to be expected, and this is due to surprising and previously undescribed synergistic effects. This pronounced effect is based on a specific and coordinated enrichment of this combination of substances, and neither is said effect thus described in the prior art, nor can the enrichment according to the invention of the substances mentioned be derived from the prior art in order to achieve the unexpectedly strong effect.

### DESCRIPTION OF THE INVENTION

The use of the preparation according to the invention as claimed in claim 1 for oral intake is aimed at not only providing the micronutrients and macronutrients required for wound healing, but also specifically supporting local and systemic processes. Therefore, the basic preparation used is a customary mixture of vitamins and nutrients that is guided by the nutritional recommendations for healthy people.

It has been found that a proportion of magnesium, zinc, vitamin B9, vitamin B12 and vitamin C (“active complex”) that is increased in a particular and coordinated manner compared to the nutritional recommendations has an unexpectedly positive effect on wound healing. The literature does state that these individual substances have various positive effects at the level of the building blocks of cells, at the level of the cells themselves and at the level of the entire organism, which effects can also promote wound healing. However, combination of the substances present in increased proportions in connection with embodiments dealt with in the dependent claims has yielded unexpectedly positive effects which have a surprisingly supportive impact at all levels of the natural wound healing processes.

The basic preparation used according to the invention as claimed in claim 1 contains the substances and amounts listed in Table 2 per gram of the basic preparation. For guidance, Table 1 presents the recommended amounts for daily intake according to Commission Directive 2008/100/EC of Oct. 28, 2008. Some of the substances are used for basic supply in order to avoid certain supply deficiencies that limit wound healing (“basic supply”). In the case of the substances of the actual active complex, the coverage of the recommendations from Table 1 per gram of the basic preparation is increased by a factor of 1.2 to 5.8 compared to the substances of the basic supply in order to achieve the surprising, particularly positive effect. One gram of the basic preparation therefore covers 30% to 35% of the respective recommendations according to Table 1 in the case of the basic supply, but 40% to 195% of the respective recommendation for the substances of the active complex.

TABLE 1
Daily recommended amounts for intake according to
Commission Directive 2008/100/EC of Oct. 28, 2008

| | | Recommended | |
|---|---|---|---|
| Name | Common name | daily intake | |
| Thiamine | Vitamin B1 | 1.1 | mg |
| Riboflavin | Vitamin B2 | 1.4 | mg |
| Niacin | Vitamin B3 | 16 | mg |
| Pantothenic acid | Vitamin B5 | 6 | mg |
| Pyridoxine | Vitamin B6 | 1.4 | mg |
| Biotin | Vitamin H | 50 | μg |
| Folic acid | Vitamin B9 | 200 | μg |
| Cobalamin | Vitamin B12 | 2.5 | μg |
| Ascorbic acid | Vitamin C | 80 | mg |
| Magnesium | | 375 | mg |
| Zinc | | 10 | mg |

TABLE 2
Composition of the basic preparation, consisting of a basic
supply and increased proportions of the active complex

| | | | | Coverage of recommended |
|---|---|---|---|---|
| | | Amount per g of | | amounts for daily intake |
| Name | Common name | basic preparation | | per g of basic preparation |
| Basic supply | | | | |
| Thiamine | Vitamin B1 | 330-385 | μg | 30-35% |
| Riboflavin | Vitamin B2 | 420-490 | μg | 30-35% |
| Niacin | Vitamin B3 | 4.8-5.6 | mg | 30-35% |
| Pantothenic | Vitamin B5 | 1.8-2.1 | mg | 30-35% |
| acid | | | | |
| Pyridoxine | Vitamin B6 | 420-490 | μg | 30-35% |
| Biotin | Vitamin H | 15-18 | μg | 30-35% |
| Active complex | | | | |
| Folic acid | Vitamin B9 | 80-390 | μg | 40-195% |
| Cobalamin | Vitamin B12 | 1.0-4.9 | μg | 40-195% |
| Ascorbic acid | Vitamin C | 32-156 | mg | 40-195% |
| Magnesium | | 150-731 | mg | 40-195% |
| Zinc | | 4-20 | mg | 40-195% |

The basic preparation can contain both the (pure) substances listed in Table 2 and compounds thereof, though in the case of compounds, only the weight fraction of the pure substance contained is taken into account in each case for ascertaining the amount per gram of basic preparation for the data in Table 2. The weight difference between the amounts specified in column 3 of Table 2 and one gram corresponds to the weight of the counter ions, for example the minerals, the molecules that may be additionally connected to the pure substances, such as complexing agents for example, and other auxiliaries without a therapeutic effect that may be required. In various embodiments, this basic preparation can be configured particularly advantageously by addition of further substances that are helpful for wound healing in combination with the basic preparation. For these preparations, what follows is specification of the amounts additionally added in relation to the basic preparation contained in the overall preparation, i.e., the amount which is additionally contained in the overall preparation per gram contained of the basic preparation.

In a further particularly preferred embodiment of the preparation used, it additionally contains 3 mg to 200 mg of tryptophan (pure or measured as the weight of the tryptophan in compounds thereof) per gram contained of the basic preparation.

In a further preferred embodiment of the preparation used, it additionally contains 0.1 g to 4 g of arginine (pure or measured as the weight of the arginine compounds thereof) per gram contained of the basic preparation.

In a further preferred embodiment of the preparation used, it additionally contains 0.1 g to 4 g of glutamine (pure or measured as the weight of the glutamine in compounds thereof) per gram contained of the basic preparation.

In a further embodiment of the preparation used, it additionally contains 3 g to 30 g of protein in the form of proteins, peptides, amino acids or a mixture thereof per gram of the basic preparation in order to provide sufficient proteins for tissue regeneration. Wound healing is a continuous process. However, nutrient supply only occurs during meals or during ingestion of the preparation and thus occurs in a discontinuous manner. This embodiment reduces the risk of phases of reduced protein availability that occur between meals in the case of patients with existing wounds because of their increased protein requirements, which phases can lead to delays in the complex wound healing process and thereby increase the risk of complications, for example due to infections. In a particularly preferred embodiment, the protein of the preparation used therefore consists of a mixture of

a. protein sources of slow availability for the body from the family of whey proteins (e.g., casein), hen's egg proteins (whole egg or egg white) or plant proteins (e.g., soy or pea proteins) having a protein absorption rate of less than 7 g/h and

b. protein sources of rapid availability from the family of whey proteins (e.g., whey), partially predigested proteins (yeast hydrolysates or other protein hydrolysates) or peptides or amino acids having a protein absorption rate of more than 7 g/h.

In a very particularly preferred embodiment, the proportion of the protein sources of slow availability is between 50% and 90% of the total protein proportion of the preparation used.

Although the amino acids alanine, glycine and cysteine can be synthesized by the body, supplementation proves to be advantageous in combination with the preparation used that is described here. Therefore, in a further preferred embodiment, what are additionally added to the protein contained in the preparation used are, in each case, between 0.1 mg and 10 g of said amino acids per gram of the basic preparation contained-individually or in any combination, as pure amino acid or as compound having a high proportion of the amino acid. Here, peptides are an example. The amounts of said amino acids additionally added are to be considered independently of one another. Particular preference is given to enrichment of all three amino acids.

In another particularly preferred embodiment, the preparation used is intended for preventing wounds or wound healing complications before a wound forms or for avoiding recurrences and therefore does not require a separate addition of protein.

In a further advantageous embodiment of the invention, the preparation used contains between 0.1 and 3 g of sources of omega-3-rich fatty acids per gram of the basic preparation contained. Firstly, they serve as an alternative energy source in order to avoid the metabolism of contained proteins for energy generation. In contrast to a high carbohydrate content, the risk of an excessive rise in the blood sugar level in diabetics is avoided at the same time. Secondly, the fatty acids have a generally positive influence on the patients' cardiovascular situation and support the local wound healing processes in a systemic manner by improvement of the flow properties of the blood, vascular dilations and their anti-inflammatory effect. Said sources of omega-3-rich fatty acids can be individual or multiple substances from the family of vegetable oils (e.g., linseed oil, . . . ), fish oils (e.g., oils prepared from herring, mackerel, salmon or sardine), algae preparations or even isolated fatty acids. In a particularly advantageous embodiment of the invention, it contains between 50 and 750 mg of eicosapentaenoic acid (EPA) and between 50 and 750 mg of docosahexaenoic acid (DHA) per gram of the underlying basic preparation, used directly as pure substance or as constituent of the aforementioned fatty acid sources.

In a further advantageous embodiment of the invention, the preparation used contains between 0.5 and 5 g of one or more blood sugar-regulating substances per gram of the basic preparation contained. The substances can be, for example, substances from the family of cyclodextrins, b-glucans or modified methylcelluloses (e.g., hydroxypropylmethylcellulose or HPMC for short). Many elderly patients with relatively large wounds suffer from diabetes mellitus. Normal control of the blood sugar level is disrupted in said patients. With all treatment options, what occurs in said patients after a meal is a rise in the blood sugar level to a value which can significantly disrupt or interrupt the normal wound healing processes. The addition described can delay the intestinal absorption of sugars and, in the case of diabetics, reduce the blood sugar peaks after a meal that are detrimental to wound healing. In a particularly advantageous embodiment, the mass fraction of said substances is between 5% and 20% of the total mass of the preparation for one portion.

In a preferred embodiment, the oral intake of the preparation used is offered in a directly ingestible form, for example in the form of a tablet or capsule or a combination thereof.

In a further preferred embodiment of the preparation used, it is made ready before oral ingestion, for example by mixing with solid or liquid foodstuffs, and thus ingested. In the context of this invention, what is explicitly also to be regarded as a liquid foodstuff is any form of beverage. This embodiment can be, for example, in the form of tablets, capsules, granules or a combination thereof.

In a further advantageous embodiment, the preparation used is offered in the form of two separate components. The first component substantially contains the aforementioned constituents which support the wound healing processes. The second component is offered separately and is used to give the finished preparation additional flavors and to thus quickly offer different flavors. However, the use of the second component does not preclude the use of flavorings in the first component. In a further advantageous embodiment, the two components have a pronounced color contrast, the intention of this being to stimulate the patients' appetite.

All of the above-described embodiments of the preparation used according to the invention can contain additional carbohydrate or fiber sources and also other auxiliaries and flavorings. They are not subject matter of this invention, but their presence or absence in the preparation does not in any way prevent the claims presented here from being fulfilled.

1. A method of nutrition-based support of the body's own wound healing processes at the local and systemic level by oral intake of a preparation, which comprises, per gram as a basic preparation, the following mixture of vitamins and nutrients:
a. 330-385 μg of vitamin B1,
b. 420-490 μg of vitamin B2,
c. 4.8-5.6 mg of vitamin B3,
d. 1.8-2.1 mg of vitamin B5,
e. 420-490 μg of vitamin B6,
f. 15-18 μg of vitamin H,
g. 80-390 μg of vitamin B9,
h. 1.0-4.9 μg of vitamin B12,
i. 32-156 mg of vitamin C,
j. 150-731 mg of magnesium,
k. 4-20 mg of zinc and
l. 3-200 mg of tryptophan.

2. The method as claimed in claim 1, wherein the preparation additionally contains 0.1 g to 4 g of arginine (pure or measured as the weight of the arginine in compounds thereof) per gram of the basic preparation.

3. The method as claimed in claim 1, wherein the preparation additionally contains 0.1 g to 4 g of glutamine (pure or measured as the weight of the glutamine in compounds thereof) per gram of the basic preparation.

4. The method as claimed in claim 1 wherein the preparation additionally contains 3 g to 30 g of protein in the form of proteins, peptides, amino acids or a mixture thereof per gram of the basic preparation.

5. The method as claimed in claim 1 wherein the protein of the preparation consists of the protein sources of slow availability for the body from the family of whey proteins, hen's egg proteins or plant proteins and protein sources of rapid availability from the family of whey proteins, partially predigested proteins or peptides or amino acids and/or a mixture of said protein sources.

6. The method as claimed in claim 1 wherein the proportion of the protein sources of slow availability in the preparation is between 50% and 90% of the total protein proportion of the preparation.

7. The method as claimed in claim 4, wherein, irrespective of the protein used, the preparation additionally contains, in each case, between 0.1 g and 10 g of the amino acid alanine or compounds thereof per gram of the basic preparation.

8. The method as claimed in claim 4, wherein, irrespective of the protein used, the preparation additionally contains, in each case, between 0.1 g and 10 g of the amino acid glycine or compounds thereof per gram of the basic preparation.

9. The method claimed in claim 4, wherein, irrespective of the protein used, the preparation additionally contains, in each case, between 0.1 g and 10 g of the amino acid cysteine or compounds thereof per gram of the basic preparation contained.

10. The method as claimed in claim 1, wherein the preparation additionally contains 0.1 g to 3 g of one or more sources of omega-3-rich fatty acids from the family of vegetable oils, fish oils, algae preparations or isolated fatty acids per gram of the basic preparation.

11. The method as claimed in claim 10, wherein the preparation contains between 50 and 750 mg of EPA and between 50 and 750 mg of DHA per gram of the basic preparation.

12. The method as claimed in claim 1, wherein the preparation additionally contains 0.5 to 5 g of one or more substances from the family of carbohydrate absorption-slowing substances consisting of beta-glucans, cyclodextrins or modified methylcelluloses per gram of the basic mixture.
